(19) United States
(12) Patent Application Publication
(10) Pub. No.: US 2026/0207869 A1
(43) Pub. Date: Jul. 23, 2026

(54) Respiratory therapy apparatus delivering intermittent positive pressure breathing assistance with a pressure plateau

(71) Applicant: EOVE, PAU (FR)

(72) Inventors: Cedric JOURDAIN, PAU (FR); Tingting LIU, LIU (FR); Marie-Amedee GALBRUN, PAU (FR); Manuel TEILLET, PAU (FR)

(21) Appl. No.: 19/450,045

(22) Filed: Jan. 15, 2026

(30) Foreign Application Priority Data
Jan. 23, 2025 (FR) 2500692

(51) Int. Cl. A61M 16/00; A61M 16/20

(52) U.S. Cl. CPC A61M 16/024; A61M 16/0003; A61M 16/201; A61M 2016/0033; A61M 2202/0007; A61M 2202/02; A61M 2205/10; A61M 2205/3334; A61M 2205/3344; A61M 2205/3351; A61M 2205/50

(57) ABSTRACT

The invention relates to a respiratory therapy apparatus (1) for supplying intermittent positive pressure breathing (IPPB) assistance to an individual, comprising a motorized turbine (2); a gas circuit (3) with a flowrate sensor (5) and a nonreturn device (6); an external patient circuit (10) for conveying the respiratory gas; a proximal-pressure sensor (7) for measuring the pressure in the patient circuit (10); and operating means (4) for commanding the turbine (2) as a function of pressure and flowrate measurements. The operating means (4) are configured to, during each inspiratory phase, command the turbine to supply a regulated gas flowrate (Q_{adjusted}), over the course of a non-zero gas insufflation duration (Di) that is sufficient to reach a set maximum-pressure threshold (Pₘₐₓ), and, when a pre-set maximum-pressure threshold (Pₘₐₓ) is reached, command the turbine to keep the external patient circuit at a plateau pressure (Pₚₗₐₜ) equal to the maximum pressure (Pₘₐₓ), over the course of a non-zero plateau duration.

### TECHNICAL FIELD

The invention relates to a respiratory therapy apparatus which delivers intermittent positive pressure breathing (IPPB) assistance, also referred to as “IPPB apparatus”, with the implementation of a pressure plateau at the end of inspiration, i.e. maintaining a constant pressure over the course of a pre-set (non-zero) plateau duration.

### BACKGROUND

In general, providing respiratory therapy of the IPPB type (hereinafter referred to as “IPPB therapy”) to certain individuals, also known as “patients”, who are suffering from respiratory problems or conditions, notably makes it possible to improve:
bronchial drainage by delivering a high inspiratory volume that makes it possible to increase the effectiveness of the expiratory flow and of coughing;
the respiratory function by exceeding the patient's maximum inspiration and thus increasing their vital capacity; and
lung recruitment, particularly in those regions of the lungs that are poorly ventilated or unventilated.

For example, EP4079356 teaches an apparatus for assisting with coughing, enabling gas insufflation to be given to patients suffering from respiratory problems who need help voiding their pulmonary secretions and/or to improve the elasticity of their lungs and thorax, whether these patients are adult or paediatric patients, and which apparatus can be used with ease by an individual having little or no medical knowledge, typically a carer, someone from the patient's family circle, also known as “the helpers”.

Other respiratory therapy apparatuses are described in DE102014001218, WO2015/110098, EP1502619 and US2013/047983.

In general, IPPB therapy relies on the application of a gas flowrate that is constant during inspiration until a given maximum pressure is reached, followed by expiration with or without positive expiratory pressure (PEP). Synchronization with the respiratory phases of the patient is achieved by detecting the effort made by the patient, typically the demand for gas at the start of inspiration, or by using a mechanism or device for manually triggering inspiration, for example by pressing a button or a command key.

There are many types of respiratory therapy devices or apparatus that allow IPPB therapy to be given, but they all have disadvantages of varying levels of severity.

At the present time, IPPB therapy devices operate on the basis of regulating the inspiration flowrate, namely the flowrate of gas (e.g. air) supplied to the airways of the patient while they are being treated.

The flowrate supplied by the gas source, such as a turbine or the like, of the gas-supplying device or apparatus is usually constant during each of the patient's inspiratory phases, leading to a flowrate curve that is rectangular in shape. Keeping the supplied flowrate constant allows the patient's lungs to fill gradually with a simultaneous gradual increase in the gas pressure in the airways, particularly in the lungs, of the patient.

The/each inspiratory phase of the patient ends when a pre-set maximum pressure, i.e. an adjustable maximum-pressure threshold, is reached. In other words, to end inspiration (i.e. an inspiratory phase), use is made of the pressure threshold that has been adjusted by the user, typically someone from the medical-care team, and that, when reached, triggers a switch to expiration (i.e. an expiratory phase). This approach aims to achieve better control over the maximum cutoff pressure so as to maximize patient comfort and limit the risks of barotrauma.

In general, the objective of the treatment session is to obtain a maximum volume in the patient's lungs, and increasing the cut-off pressure is one way of achieving this.

However, it has been found in practice that the effectiveness of the treatment and/or the comfort of the patients, as well as adherence to the treatment, notably depend on the physiology of the patients themselves, notably in terms of compliance and resistance, but also on the type of breathing interface used for administering the gas, i.e. breathing mask with or without leakage.

One problem addressed is therefore that of providing a respiratory therapy apparatus or device for delivering intermittent positive pressure breathing assistance, that is to say therapy of the IPPB type, or “IPPB apparatus”, which is improved, notably which makes it possible to overcome all or some of the abovementioned drawbacks, in particular for delivering more effective IPPB therapy with increased patient comfort and/or better compliance, preferably irrespective of the physiology of the patient in question (e.g. compliance and resistance), and/or take better account of the features of the type of respiratory interface used and/or of the accessories likely to be used during the treatment, such as a nebulizer, a filter, etc.

### SUMMARY

One solution according to the invention is a respiratory therapy apparatus (i.e. device or installation), namely an “IPPB apparatus”, for supplying intermittent positive pressure breathing (IPPB) assistance to an individual, i.e. a patient, and comprising:
a peripheral casing or shell,
a motorized turbine for supplying a respiratory gas under pressure,
an internal gas circuit for conveying the respiratory gas delivered by the turbine,
a flowrate sensor arranged on the internal gas circuit,
a nonreturn device arranged in the internal gas circuit downstream of the flowrate sensor,
an external patient circuit that can be fluidically connected to the internal gas circuit to convey the respiratory gas coming from the internal gas circuit,
a proximal-pressure sensor arranged to enable measurement of the pressure (i.e. to take instantaneous-pressure measurements) within the external patient circuit near to the expiratory valve, and
operating means connected to the proximal-pressure sensor and to the motorized turbine and configured to command the turbine as a function of at least one pressure measurement taken by the proximal-pressure sensor and of at least one flowrate measurement taken by the flowrate sensor.

The turbine, the operating means, the internal circuit, the flowrate sensor and the nonreturn device are arranged in the peripheral casing or shell of the apparatus, that is to say inside the apparatus.

In addition, in the IPPB apparatus, the operating means are configured to, during each of the individual's, i.e. the patient's, inspiratory phases:
a) command the turbine to supply a regulated gas flowrate (Q_{adjusted}), over the course of a non-zero gas insufflation duration (Di) that is sufficient to reach a set maximum-pressure threshold (Pₘₐₓ),
b) and, when a measured pressure (Pₘₑₐₛ) measured by the proximal-pressure sensor is (i.e. is or becomes) greater than or equal to the pre-set maximum-pressure threshold (Pₘₐₓ) (i.e. Pₘₑₐₛ=Pₘₐₓ), command the turbine to supply a plateau pressure (Pₚₗₐₜ) corresponding to the maximum-pressure threshold (Pₘₐₓ) so as to keep at least part of the external patient circuit at said plateau pressure (Pₚₗₐₜ), over the course of a given non-zero plateau duration (Dp).

In other words, according to the invention, over the course of the gas insufflation duration (Di), the turbine (i.e. its motor) is operated to control its flowrate, whereas thereafter, over the course of the plateau duration (Dp) it is operated to control its pressure.

Keeping the external patient circuit at the plateau pressure (Pₚₗₐ), which is equal to the pre-set maximum-pressure threshold (Pₘₐₓ), over the course of the plateau duration (Dp) makes it possible to increase patient comfort and/or the overall treatment effectiveness and/or improve patient compliance with their treatment.

Indeed, the pressure-plateau phase makes it possible to increase the volume of gas, i.e. air, delivered to the patient, i.e. to the individual receiving treatment, and to improve the propagation of gas in the lungs without increasing the maximum cut-off pressure, i.e. the maximum pressure delivered. Not increasing the cut-off pressure is one way of limiting barotrauma-type side effects that the treatment might have on the lungs.

In other words, the pressure-plateau phase allows a member of the medical-care team, i.e. a clinician or similar, to adapt the patient's therapy to obtain the best possible trade-off between maximum pressure and treatment effectiveness, this being in the interest of the individual treated in this way.

Depending on the embodiment concerned, the apparatus of the invention, namely the “IPPB apparatus”, may comprise one or more of the following features:
the operating means are configured to command the electric motor of the turbine to supply a desired gas flowrate and/or pressure.
the electric motor of the turbine is preferably a brushless and/or DC motor.
the operating means are configured to command the turbine, i.e. its motor, so that the regulated gas flowrate (Q_{adjusted}) supplied in step a) is an (approximately) constant or decreasing flowrate over the course of the gas insufflation duration (Di). Of course, the flowrate may exhibit slight variations or fluctuations around the desired flowrate value, for example of about +/−5%.
it comprises pressure-adjusting means configured to allow the user to adjust the plateau duration (Dp).
as a preference, the plateau duration (Dp) is comprised between 0.1 and 10 sec, preferably between 0.5 and 5 sec.
it comprises pressure-adjusting means configured to allow the user to adjust the maximum-pressure threshold (Pₘₐₓ).
as a preference, the maximum-pressure threshold (Pₘₐₓ) is comprised between 10 and 50 cmH2O.
it comprises flowrate adjusting means to allow the user to adjust the desired flowrate value (Q_{adjusted}).
with preference, the flowrate value (Q_{adjusted}) is comprised between 1 and 200 L/min, preferably between 1 and 150 L/min, and more preferably still between 5 and 100 L/min.
the gas flowrate (Q_{adjusted}) supplied over the course of the gas insufflation duration (Di) is preferably constant or approximately constant.
the duration-adjusting means and/or the pressure-adjusting and/or flowrate-adjusting means comprise one or more buttons, keys or the like, particularly one or more buttons displayed on a touchscreen.
the operating means are configured to compare a measured proximal-pressure value (Pₘₑₐₛ) measured by the proximal-pressure sensor with the set maximum-pressure threshold (Pₘₐₓ) and to trigger step b) when the measured pressure value (Pₘₑₐₛ) measured by the proximal-pressure sensor (7) is or becomes equal to the pre-set maximum-pressure threshold (Pₘₐₓ), i.e. Pₘₑₐₛ=Pₘₐₓ.
the pressure adjustment in step b) is used to keep at least part of the external patient circuit at the plateau pressure (Pₚₗₐ), over the course of a given non-zero plateau duration (Dp).
an expiratory valve is arranged on the external patient circuit.
at the end of the plateau duration (Dp), the expiratory valve opens so as to discharge to the atmosphere at least some of the gas pressure present in the external patient circuit.
the expiratory valve opens in response to a drop in pressure in the internal gas circuit at the end of the plateau duration (Dp).
a pneumatic operating line for operating the expiratory valve is fluidically connected to the internal gas circuit between the turbine and the flowrate sensor.
the pneumatic operating line collaborates with the expiratory valve to control the opening or closing of said expiratory valve.
the opening of the expiratory valve enables gas, i.e. gas pressure, to be discharged from the external patient circuit to the ambient atmosphere and the pressure in all or part of the external patient circuit to decrease.
a pneumatic operating line is a pneumatic connection, typically a flexible tube, preferably made of polymer.
the operating means are connected to the proximal-pressure sensor, namely the pressure sensor may, depending on the embodiment concerned, be electrically connected to said operating means and/or (directly) integrated in said operating means, so as to collaborate with said operating means.
as a preference, the proximal-pressure sensor is integrated in the operating means, particularly borne by an electronic board that forms part of the operating means.
the proximal-pressure sensor comprises a pressure measurement line that is connected to the patient circuit downstream of the expiratory valve.
the proximal-pressure sensor is configured to enable measurement of the pressure within the external patient circuit between the expiratory valve and a breathing interface providing the gas exchanges with the patient, typically during the patient's inspiratory phases and/or possibly during the patient's expiratory phases.
the internal gas circuit comprises a passage, a duct or the like serving to carry the gas.
the breathing interface is a breathing mask, a tracheotomy tube or a mouthpiece.
the internal gas circuit comprises a gas outlet.
the external patient circuit is fluidically connected to said gas outlet of the internal gas circuit.
the external patient circuit comprises a flexible tube, typically made of polymer.
the external patient circuit may also comprise or employ additional fluidic-connection elements such as connectors, couplings or the like.
the external patient circuit comprises a single branch or is a single-branch circuit.
according to another embodiment, the external patient circuit may be a dual-branch circuit.
the operating means comprise at least one microprocessor.
said at least one microprocessor is arranged on at least one electronic board.
the operating means comprise at least one electronic board.
the motorized turbine comprises an electric motor.
the electric motor is electrically powered (110/220V).
the operating means are configured to command the accelerations of the electric motor of the turbine and/or the decelerations or brakings of said motor. Deceleration means a braking or slowing of the motor, achieved for example by inverting the phases of the motor.
the nonreturn device comprises a check valve, a nonreturn valve or the like.
the nonreturn device is configured to oppose any backflow, namely any flow back towards the turbine, i.e. flowing in the opposite direction to the normal direction of circulation of the gas from the turbine towards the patient.
the expiratory valve is preferably an at least 2-way valve.
according to another embodiment, the expiratory valve may be a PEP valve.
the electric motor of the turbine is operated, i.e. commanded or controlled, by the operating means.
the pneumatic operating line comprises a flexible tube made of polymer and of small diameter, typically approximately 2 to 10 mm in diameter.
it comprises a peripheral casing or shell formed from one or more sub-parts fixed together, for example by screw-fastening.
the peripheral casing or shell may be made of rigid polymer.
it comprises a graphical user interface or GUI.
the GUI may comprise a display screen, preferably of the touchscreen type, and/or selection or validation keys, notably touch-sensitive keys displayed on the screen.
it comprises memory-storage means, such as a RAM or EEPROM type memory, or a flash memory or the like.
the operating means command the start of a slowing down or braking of the turbine when said operating means determine, based on the measured proximal pressure, typically as close as possible to the patient, the end of the gas insufflation phase (Di).
the operating means are configured to compare the measured proximal-pressure value measured by the proximal-pressure sensor with the (pre-)adjusted maximum-pressure value, namely the maximum-pressure threshold (Pₘₐₓ), and from this to deduce the end of the gas insufflation duration (Di) and/or the start of the plateau duration (Dp).
the operating means are configured to trigger step b) in response to the detection of a proximal pressure (i.e. an instantaneous pressure) equal to the maximum pressure threshold (Pₘₐₓ).
the motor of the turbine is configured to be able to rotate at a rotational speed of as much as 50,000 rpm, or even 70,000 rpm.
the operating means comprise a timer enabling the durations to be determined or measured.
the maximum-pressure threshold (Pₘₐₓ) is stored in the memory-storage means.
the maximum-pressure threshold (Pₘₐₓ) is (pre-)set, i.e. adjusted by the user (i.e. medical personnel), preferably via the GUI.
the given non-zero plateau duration (Dp) is stored in the memory-storage means.
the given non-zero plateau duration (Dp) is (pre-)set, i.e. adjusted by the user (i.e. medical personnel), preferably via the GUI.
the expiration valve is configured to open automatically as soon as the turbine starts to decelerate (i.e. brake), i.e. that is to say when the (motor of the) turbine slows down, typically after the pressure-plateau phase. This is possible by virtue of the fluidic connection of the expiration valve operating line downstream of the turbine and upstream of the nonreturn device, namely between the turbine and the gas nonreturn device, and the pneumatic operation (i.e. control by pressure) of said expiration valve by said operating line.
the expiration valve comprises a valve body comprising flexible sealing means.
the flexible sealing means comprise a membrane, a balloon or the like, preferably flexible and/or deformable, typically a membrane.
the pneumatic operating line controls the opening and/or the closing of the expiration valve by acting pneumatically, namely by supplying pressurized gas, on the flexible sealing means of the valve body.
the valve body of the expiration valve comprises at least one atmospheric-venting orifice.
the flexible sealing means are configured to allow or, conversely, to prevent the circulation of the flow of gas through the atmospheric-venting orifice, namely the discharging of gas to the atmosphere.
the flexible sealing means are arranged on a passage for a flow of gas passing through the valve body.
the flexible sealing means are configured to collaborate with at least part of the wall of the valve body so as to control the flow of gas or seal against the flow of gas, particularly in at least part of the wall of the gas flow passage arranged in the valve body.
the internal gas circuit comprises a gas outlet, for example borne by an outlet connector or endpiece or the like.
the external patient circuit is fluidically connected to the gas outlet of the internal gas circuit, for example is connected to the outlet connector or endpiece or the like of the internal gas circuit.
over the course of the plateau duration Dp, the flowrate delivered by the turbine gradually decreases.
a residual gas flowrate is maintained over at least some of the course of the plateau duration Dp.
over the course of the plateau duration Dp, the expiratory valve is closed so as to keep at least part of the external patient circuit at the plateau pressure Pₚₗₐ.
at the end of the plateau duration Dp, the expiratory valve opens so as to discharge at least some of the gas pressure present in the external patient circuit.
the expiratory valve is kept closed over the course of the inspiration duration Di and the plateau duration Dp.

Furthermore, the invention also relates to a method for supplying respiratory therapy of the IPPB type to an individual who needs it, i.e. a patient, wherein use is made of a respiratory therapy apparatus (i.e. device or installation), as described above or below, to supply a respiratory gas to said individual, which method comprises, during each of the individual's inspiratory phases:
i. supplying a regulated gas flowrate (Q_{adjusted}), over the course of a non-zero gas insufflation duration (Di) that is sufficient to reach a set maximum-pressure threshold (Pₘₐₓ), and
ii. when a measured pressure (Pₘₑₐₛ) measured by the proximal-pressure sensor is greater than or equal to the pre-set maximum-pressure threshold (Pₘₐₓ), supplying a plateau pressure (Pₚₗₐₜ) corresponding to the maximum-pressure threshold (Pₘₐₓ) so as to keep at least part of the external patient circuit at said plateau pressure (Pₚₗₐₜ), over the course of a given non-zero plateau duration (Dp). In steps i) and ii), the gas flowrate and the gas pressure are supplied by the turbine of the apparatus which is operated by the operating means of the apparatus.

In the context of the present invention:
a “pressure sensor” is the name given to any device or means able to determine a gas pressure.
a “flowrate sensor” is the name given to any device or means able to determine a gas flowrate, including indirectly via pressure measurements.
the terms “apparatus” and “device” are considered equivalent and interchangeable.
the terms “turbine”, “compressor”, “blower” or the like are considered equivalent and interchangeable.
the terms “valve” and “valve unit” are considered equivalent and interchangeable.
the terms “means” and “device” are considered equivalent and interchangeable, for example the terms “operating means” are considered equivalent to and interchangeable with the terms “operating device”.
the terms “operated”, “commanded” and “controlled” are considered equivalent and interchangeable.
the terms “operate”, “command” and “control” are considered equivalent and interchangeable.
an individual's breathing is considered to comprise or to be formed of successive respiratory cycles, each respiratory cycle comprising an inspiratory phase followed by an expiratory phase.
over the course of an inspiratory phase, an individual inhales/draws into their lungs a respiratory gas, such as air or an air/O2 mixture.
over the course of an expiratory phase, an individual exhales/expels from their lungs a gas containing CO2.

### BRIEF DESCRIPTION OF VIEWS OF DRAWINGS

The invention will now be better understood from the following detailed description, provided by way of non-limiting illustration, with reference to the appended figures, in which:

FIG. 1 is a process diagram of one embodiment of a respiratory therapy apparatus according to the invention.

FIG. 2 schematically depicts (in a partial exterior view) one embodiment of an apparatus according to the invention, such as that of FIG. 1, and its connection to the mains.

FIG. 3 schematically depicts the flowrate (Q) curve and pressure (P) curve obtained with an IPPB apparatus according to the invention.

### DETAILED DESCRIPTION

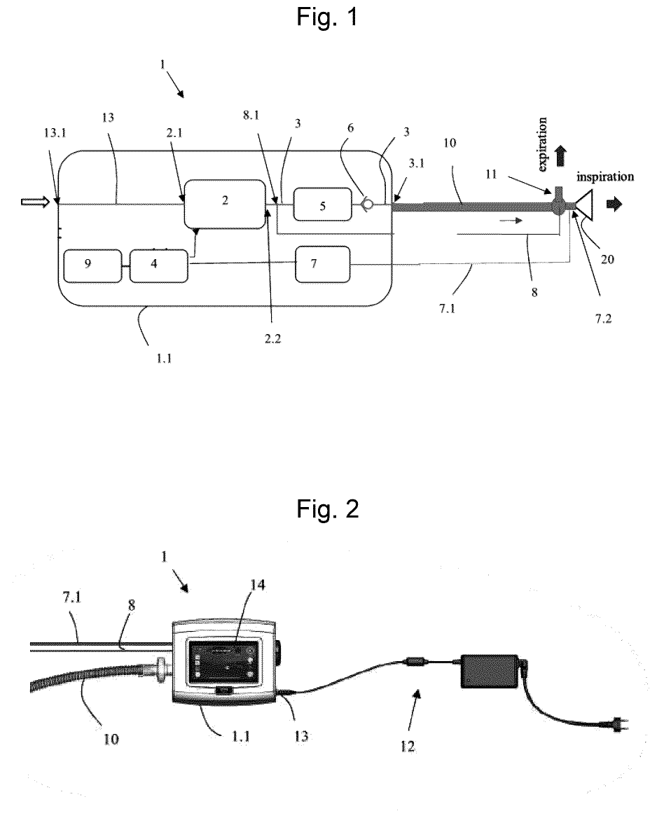

FIG. 1 and FIG. 2 schematically depict one embodiment of a respiratory therapy apparatus or device 1 according to the invention, making it possible to provide intermittent positive pressure breathing assistance, hereinafter referred to as “IPPB therapy”, to an individual who needs it, namely typically to a patient who is to be treated, or the like.

As illustrated in the process diagram of FIG. 1, the respiratory therapy apparatus 1 or “IPPB apparatus” comprises a motorized turbine 2 (also referred to as (micro) blower, pump, compressor or the like) combined with a passive command system comprising an expiration valve 11, also referred to as an expiratory valve, and with proximal-pressure measuring means, typically a proximal-pressure sensor 7, as described hereinafter, with a view to delivering the IPPB therapy to the patient effectively and with improved patient comfort, notably in terms of limiting or reducing annoying noise, namely the noise generated during the functioning of the apparatus 1.

More specifically, the motorized turbine 2 comprises an electric motor, typically of the brushless DC type, which is operated, i.e. controlled, by operating means 4, typically by one (or more) microprocessor(s) arranged on one (or more) electronic board(s). The turbine 2 is supplied with ambient air by an air inlet line 13, such as a duct, a passage or the like, which connects an air inlet 13.1 to the inlet 2.1 of the turbine 2.

The motorized turbine 2 has a conventional architecture, namely it schematically comprises an electric motor arranged in a protective housing, which is surmounted by a volute casing comprising an internal compartment in which a bladed wheel is arranged. The bladed wheel is borne by the shaft of the motor. It is rotationally driven by the shaft of the motor, when it is functioning, so as to draw in the respiratory gas, such as air, which then enters the internal compartment of the volute casing, then re-emerges therefrom before being sent to the patient.

The turbine 2 then supplies the respiratory gas, such as air, under pressure to an internal gas circuit 3 that serves to carry, i.e. convey, the respiratory gas delivered at the outlet 2.2 of the turbine 2 to a breathing interface 20, such as a breathing mask, a mouthpiece, or a tracheotomy tube, supplying the respiratory gas to the airways of the patient who is to be treated.

When the electric motor of the turbine 2 (i.e. its rotational speed) is controlled by the operating means 4, the motor shaft bearing the bladed wheel is rotationally driven, enabling the gas to be supplied at the desired flowrate, for example at the regulated flowrate (Q_{adjusted}), which may be constant or variable depending on the control performed, as explained hereinafter.

Conversely, the operating means 4 can also control the rotational speed of the motor in order to brake or slow same. In that case, the rotational speed of the motor shaft bearing the bladed wheel decreases more or less rapidly and the supply of gas then decreases at a rate that correlates with the slowing/braking of the motor.

One example of a turbine or (micro) blower for a medical ventilation apparatus is given in EP2947328, but of course other types of turbine may be suitable.

Using a motorized turbine 2 to deliver the gas makes it possible to limit the noise generated while the apparatus 1 is functioning.

In order to convey the respiratory gas coming from the internal gas circuit 3, namely a circuit arranged inside the shell 1.1 of the apparatus 1, as far as the breathing interface 20, an external patient circuit 10 is provided, this typically being one (or more) flexible tube(s) or duct(s) fluidically connected to a gas outlet 3.1 of the internal gas circuit 3, typically a flexible tube made of polymer. The gas outlet 3.1 may be borne by an outlet coupling or connector arranged on the casing 1.1 of the apparatus 1. The external patient circuit 10 is fluidically coupled to said outlet coupling, for example by being push-fitted into it, screwed into it, or fixed in it using a bayonet system or any other suitable connection system, the flexible tube preferably comprising fluidic-connection means complementary to those of the outlet coupling. The patient circuit 10 is therefore located outside the shell 1.1 of the apparatus 1.

In order to measure the gas flowrate within the apparatus 1, a flowrate sensor 5 is arranged on the internal gas circuit 3, namely downstream of the turbine outlet 2 and upstream of the gas outlet 3.1 of the gas circuit 3. The flowrate sensor 5 supplies the flowrate measurements to the operating means 4 where they are processed in order to regulate the operation of the turbine 2, typically the accelerations or braking of the electric motor thereof. The flowrate sensor 5 is electrically connected to the operating means 4, typically by electrical connections, such as electric cables or the like, so as to transmit to it the flowrate measurements taken.

These flowrate measurements can also be used to check the correct operation of the nonreturn device 6, e.g. check valve, by ensuring that there is no backflow flowing back towards the turbine 2 during the patient's expiratory phases, namely to check that the nonreturn device 6 is providing fluidic sealing in the backflow direction.

The external patient circuit 10 for its part comprises the expiratory valve 11 which serves to control the discharges of gas exhaled by the patient and/or all or some of the gas pressure present in the patient circuit 10, particularly after the plateau phase as explained hereinafter, into the ambient atmosphere, given that this expiratory valve 11 allows the downstream part of the patient circuit 10 to be placed in communication with the outside, namely the ambient atmosphere, particularly at the end of the inspiratory phase and/or during the expiratory phases of the patient.

Moreover, in order to prevent backflow of gas/pressure and allow these to be discharged better via the expiratory valve 11, a nonreturn device 6, namely a check valve, a one-way valve or the like, is arranged on the internal gas circuit 3, downstream of the flowrate sensor 5 and upstream of the gas outlet 3.1 of the gas circuit 3. Such a nonreturn device 6 also offers the advantage of being simple to implement and of not requiring any particular operational control, and therefore also of being low in cost.

The presence of this nonreturn device 6, typically a check valve, thus makes it possible to improve the functioning of the apparatus 1, typically the opening of the expiratory valve 11 during the expiratory phases, as explained hereinbelow.

The expiratory valve 11, particularly the opening and/or closing thereof, is/are commanded or controlled by a pneumatic operating line 8, such as a flexible duct, which is fluidically connected 8.1 to the internal gas circuit 3 between the turbine 2 and the flowrate sensor 5. The pneumatic operating line 8 collaborates with the expiratory valve 11 by supplying it with a gas pressure that serves to control the opening or the closing of said expiratory valve 11, namely the extent to which it is open/closed.

As a preference, the opening and/or the closing of said expiratory valve 11 is/are controlled by flexible sealing means on which the gas pressure supplied by the pneumatic operating line 8 acts, as explained hereinbelow.

Finally, the external patient circuit 10 also comprises a proximal-pressure sensor 7 arranged in such a way as to enable measurement of the pressure, i.e. the instantaneous pressure, within the patient circuit 10 and close to 7.2 the expiratory valve 11, typically between the expiratory valve 11 and the breathing interface 20.

This proximal-pressure sensor 7 makes it possible to detect the patient's demands for gas, namely their inhalations, but also whether the set maximum pressure threshold (Pₘₐₓ) has been reached at the end of the inspiratory phrase, as detailed hereinbelow.

The proximal-pressure sensor 7 is connected to the operating means 4, namely collaborates therewith, so that the latter can operate the turbine 2 on the basis of all or some of the measurements taken by the proximal-pressure sensor 7. As a preference, the proximal-pressure sensor 7 is directly integrated in the operating means 4, for example borne by an electronic board of said operating means 4.

The use of an operationally controlled motorized turbine 2 ensures improved comfort both in terms of the lower noise generated by the apparatus 1 during an IPPB treatment and in terms of the adaptability of the profile of the flowrate delivered to the patient, for example a flowrate of “rectangular” shape or another shape.

Moreover, the measurement of the proximal pressure, using the proximal-pressure sensor 7 provides the device 1 with additional performance by virtue of the high precision of the pressure measurements taken, and also comfortable use for the patient, by virtue of better detection of the respiratory effort exerted by the patient, making it possible to obtain effective, or even (near-)optimal synchronization between the patient's demand for gas and the supply of gas by the turbine during the inspiratory phases and, conversely, the discharging of the exhaled gases via the expiratory valve 11.

The apparatus 1 also comprises adjustment or selection means, such as virtual keys displayed by the display, i.e. digital screen 14, of a graphical user interface, or

GUI, 9. The display screen 14 is preferably of the touchscreen and/or colour display screen type. It comprises selection or validation keys, notably touch-sensitive keys displayed on the screen.

The turbine 2, the operating means 4, the internal circuit 3, the flowrate sensor 5 and the nonreturn device 6 are arranged in the peripheral casing or shell 1.1 of the apparatus 1, for example a casing made of a rigid polymer or some other suitable material.

In general, electrical power supply means, such as a mains (110/220V) electrical connection are also provided, these enabling electrical power to be supplied to the apparatus 1, particularly to all the components that require electrical power in order to function, such as the operating means 4, the GUI 9, the screen 14, the sensors 5, 7, etc. As a preference, as illustrated in FIG. 2, the electrical power supply means may comprise a unit 12 comprising an electrical cable with a current transformer and a plug for connecting to the mains and that can be connected to an electrical connector 13 of the apparatus 1.

Furthermore, the apparatus 1 may also comprise means for starting and/or stopping it, such as an “On/Off” key or the equivalent.

While the apparatus 1 is functioning, during the patient's inspiratory phases, the turbine 2 is operated in such a way as to deliver a constant or decreasing, preferably constant or approximately constant, gas flowrate (Q_{adjusted}) depending on the control parameter(s) or setting(s) input, set or selected by the user via the GUI 9, particularly a maximum-pressure value, also referred to as maximum-pressure threshold (Pₘₐₓ), and possibly a maximum inspiration time (Ti), namely a maximum duration over the course of which gas is delivered to the patient during each inspiratory phase including the durations Dp and Di below.

According to other embodiments, other parameters, such as an inspiration flowrate, inspiratory and/or expiratory gradient, a trigger sensitivity, a therapy time, etc. may also be selected.

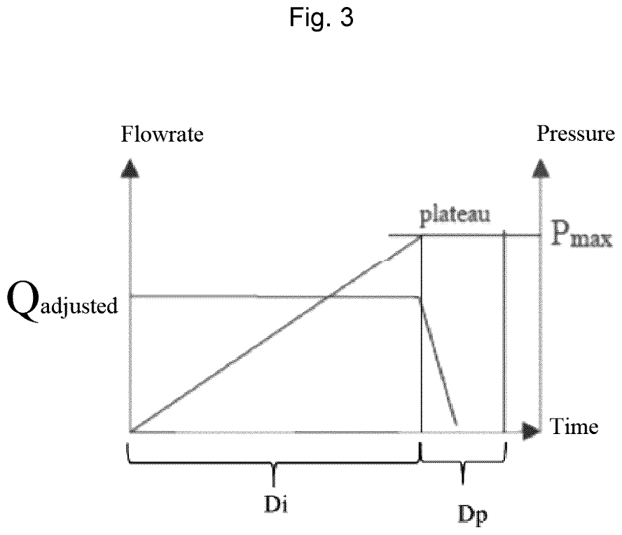

More specifically, as illustrated in FIG. 3, according to the invention, the operating means 4, typically a microprocessor, are configured to command the turbine 2, typically the electric motor of the turbine, during each of the patient's inspiratory phases, to first of all supply a controlled/desired gas flowrate (Q_{adjusted}), typically an air flowrate, which is preferably constant, over the course of a non-zero gas insufflation duration (Di) that is sufficient to achieve a pre-set maximum-pressure threshold (Pₘₐₓ), for example comprised between 10 and 50 cmH2O.

Then, when the measured instantaneous pressure (Pₘₑₐₛ) measured by the proximal-pressure sensor 7 reaches the pre-set maximum-pressure threshold (Pₘₐₓ), i.e. Pₘₑₐₛ=Pₘₐₓ, the operating means 4 stop operating the turbine 2 to control flowrate and start operating it to control pressure. At this time, the turbine 2 is no longer operated to control flowrate and slows down, thereby causing drop, which is preferably gradual, in the gas flowrate supplied.

However, as illustrated in FIG. 3, according to the invention, at least part of the external patient circuit 10 is then kept at a plateau pressure (Pₚₗₐ) equal to the pre-set maximum-pressure threshold (Pₘₐₓ), i.e. Pₚₗₐ=Pₘₐₓ, over the course of a given non-zero plateau duration (Dp), for example a plateau duration (Dp) comprised between 0.5 and 5 sec. In general, the gas insufflation duration (Di) is longer than the plateau duration (Dp).

For this, the operating means 4 command the turbine 2 to supply a plateau pressure (Pₚₗₐₜ) corresponding to the maximum-pressure threshold (Pₘₐₓ).

In other words, during each of the patient's inspiratory phases, the operating means 4 first of all command the turbine 2 to control flowrate, and then, when the plateau pressure has been reached, at the end of the gas-insufflation duration Di, they command it to control pressure over the course of the plateau duration Dp.

Throughout the duration of the patient's inspiratory phase, namely over the course of the durations Di and Dp, the expiratory valve 11 remains closed.

FIG. 3 illustrates a flowrate (Q) curve and pressure (P) curve as a function of time, during an inspiratory phase of the patient, the adjusted flowrate Q_{adjusted} adjusted by the user and delivered to the patient by the turbine 2 is kept constant over the course of the gas insufflation duration Di, for example an adjusted flowrate Q_{adjusted} comprised between 5 and 100 L/min.

The gas insufflation duration Di corresponds to the time needed for the gas pressure in the external patient circuit 10, and therefore also in the patient's lungs, to increase gradually until a desired maximum pressure, namely the pre-set maximum-pressure threshold Pₘₐₓ, is reached. In this case, the increase in gas pressure is linear; however, it could be non-linear given that the increase in pressure is the result of the gas flowrate supplied to the patient's lungs, i.e. the volume of gas entering the lungs. This increase in gas pressure can be (very) variable depending in particular on the compliance and/or resistance demonstrated by the patient's lungs.

It can be seen that, once the maximum-pressure threshold Pₘₐₓ has been reached, the gas pressure in the patient circuit 10 is kept at the maximum-pressure threshold value Pₘₐₓ so as to obtain a pressure plateau (Plateau), i.e. a plateau pressure Pₚₗₐ equal to Pₘₐₓ, prevailing in the patient circuit 10, particularly in its downstream part close to the patient and their airways.

This plateau pressure Pₚₗₐ is maintained over the course of the plateau duration Dp, whereas in the same time interval the flowrate gradually decreases from its adjusted value Q_{adjusted} to a lower value, for example a zero value or a value close to the zero value (i.e. 0 L/min). This gradual decrease in the flowrate corresponds to the slowing or braking of the turbine 2 which is now operated to control pressure (rather than flowrate) in order to maintain the plateau pressure Pₚₗₐ.

By virtue of the application of this pressure plateau (Plateau), the external patient circuit 10 is kept at the plateau pressure Pₚₗₐ which is equal to the pre-set maximum-pressure threshold Pₘₐₓ, over the course of the plateau duration Dp, the result of which is to increase patient comfort and the effectiveness of the patient's overall treatment, given that the pressure-plateau phase makes it possible to increase the volume of gas, i.e. air, delivered to the patient and thus to improve the propagation of the gas into the patient's lungs, all the while without increasing the maximum cut-off pressure, namely the maximum pressure delivered, and therefore limiting any barotrauma-type side effects of the treatment.

As already mentioned, by virtue of the pressure-plateau phase, a member of the medical-care team, i.e. a clinician or similar, can adapt the patient's therapy to obtain the best possible trade-off between maximum pressure and treatment effectiveness.

According to the invention, the operating means 4 therefore compare the proximal-pressure measurements Pₘₑₐₛ coming from the proximal-pressure sensor 7 against the pre-set maximum-pressure threshold Pₘₐₓ, and trigger the plateau phase and maintenance of the plateau pressure Pₚₗₐ, when these pressures Pₘₑₐₛ and Pₘₐₓbecome identical.

At the end of the plateau duration Dp the turbine 2 ceases to be operated for the control of pressure and there is a sharp drop in the pressure prevailing in the internal circuit 3 of the apparatus 1, particularly at the tapping 8.1 for the operating line that operates the expiratory valve 11, and therefore within the operating line that operates the expiratory valve 11 itself. This sharp drop in pressure will then cause

the expiratory valve 11 to open so as to discharge at least some of the gas pressure present in the patient circuit 10 to the external atmosphere and thus obtain a drop in pressure in the patient circuit 10 as described in detail hereinafter.

However, it should also be noted that a residual gas flowrate is maintained over at least some of the course of the plateau duration Dp. In this case, the residual flowrate gradually decreases with a linear or non-linear profile, for example a substantially parabolic or other profile.

The control parameter(s), particularly the maximum-pressure threshold (Pₘₐₓ), the plateau duration (Dp) and/or the adjusted flowrate Q_{adjusted} may be stored in memory-storage means of the apparatus 1, such as a flash memory, a RAM, an EEPROM or the like.

These parameters can be adjusted via pressure-, flowrate-and/or duration-adjusting means, such as one or more adjusting buttons or keys, for example virtual or analogue keys displayed by the GUI.

In general, as illustrated in FIG. 1, the gas coming from the turbine 2 while it is functioning is delivered to the patient after having been conveyed by the internal gas circuit 3, and therefore via the flowrate sensor 5 and the nonreturn device 6 (i.e. check valve or the like).

The pneumatic operating line 8, i.e. flexible tube or the like, that serves to operate the expiratory valve 11 is connected to the internal gas circuit 3 downstream (at 8.1) of the outlet of the turbine 2, which means that the expiratory valve 11 can be closed typically by action on the flexible sealing means of the expiratory valve 11 while the patient is inhaling, namely over the course of the inspiration and/or plateau phases (i.e. over the course of the durations Di and/or Dp), by virtue of the gas pressure coming from the turbine 2 that travels via the pneumatic operating line 8 as far as the expiratory valve 11.

As a preference, the flexible sealing means of the expiratory valve 11 comprise a balloon or a flexible/deformable membrane or the like, and an atmospheric-venting orifice or vent in communication with the atmosphere so as to control the flow of gas, namely to block any gas from leaving via said atmospheric-venting orifice thereby preventing any gas from leaving during the inspiratory phases, or conversely to

uncover this orifice to a greater or lesser extent so as then to allow gas to pass to the atmosphere and therefore to leave the external patient circuit 10. Such an architecture is conventional.

In other words, the flexible sealing means are arranged in the valve body and collaborate with part of the wall of said valve body in the manner of a valve shutter and valve-shutter seat so as to control the flows of gas through the valve body and the release of these flows of gas to the atmosphere.

In one embodiment, the expiratory valve 11, i.e. expiration valve, may further comprise a fluidtight zone comprised between the membrane and the upper body of the valve unit 11 which allows operation as a function of the pressure sent to the fluidtight zone via the operational-control tapping.

Moreover, the proximal-pressure measurement(s) taken by the proximal-pressure sensor 7 reflects (reflect) the pressure prevailing in the patient circuit 10 as close as possible to the patient (at 7.2), namely in the immediate vicinity of the patient's airways.

When the proximal pressure measured as close as possible to the patient reaches the (pre-)adjusted maximum pressure value, namely the pre-set maximum-pressure threshold (Pₘₐₓ), or when a (pre-)set or adjusted maximum inspiration time has been reached, the operating means 4 control the turbine 2, more specifically the electric motor thereof, so that this turbine slows and/or brakes, namely so that the rotations of the motor 2 decrease and/or stop, but in such a way as to keep a positive pressure in the circuit 10 at the plateau-pressure value Pₚₗₐ.

As has already been stated, the comparisons between the measured proximal-pressure and pre-set maximum-pressure, namely the pre-set maximum-pressure threshold (Pₘₐₓ), values are made by the operating means 4, typically by a microprocessor.

Moreover, the operating means 4 may further comprise a timer or the like for determining the durations or other time periods.

Because of the presence of the nonreturn device 6, any reduction or drop in pressure is not transmitted directly to the patient circuit 10 but remains in the internal circuit 3 and in the operating line 8 that operates the expiration valve 11, thereby allowing the valve 11 to open, after the plateau phase, since the pressure prevailing in the balloon or on the membrane of the valve 11 decreases, thus allowing gas to escape to the atmosphere, and therefore allowing the patient to exhale through said expiratory valve 11 which then is in fluidic communication with the atmosphere, via its atmospheric-venting orifice, given that the balloon or the membrane is no longer sealing against the passage of gas.

Patient expiration therefore progresses passively through the expiration valve 11 of the patient circuit 10 and to the atmosphere, i.e. through the valve body and the atmospheric-venting orifice.

More generally, in the apparatus of the invention, the use of the proximal pressure for synchronizing the patient's inspiratory and expiratory phases allows the system better selectivity which is no longer influenced by the flowrate coming from the turbine 2 and by the pneumatic resistances of the patient circuit 10.

The apparatus 1 detects the patient's respiratory effort more precisely and with greater sensitivity, making it possible to improve the IPPB treatment notably as a result of better synchronization between the delivery of gas and the patient's respiratory effort.

On examining FIG. 3, it can be seen that, during inspiration, the turbine 2 first of

all delivers the desired gas flowrate Q_{adjusted} which can be decelerating or constant, specifically in this case constant, depending on the adjustments made by the user via the interface or GUI.

The flowrate Q_{adjusted} is delivered to the patient through the flowrate sensor 5 and the check valve 6. The flowrate Q_{adjusted} is regulated using closed-loop control by the operating means 4, such as a computer, on the basis of flowrate measurements taken at the outlet/downstream of the turbine 2. The speed of the turbine 2 can thus be adapted by the operating means 4 to suit the desired flowrate profile. The regulating control, for example of the proportional integral derivative (PID) type, makes it possible to maintain a minimum difference between the desired flowrate profile and the flowrate profile resulting from the adaptation of the turbine speed and to do so irrespective of the resultant pressure provided that this pressure remains below the maximum-pressure threshold Pₘₐₓ at which inhalation is cut off.

By virtue of the pressure plateau (plateau), a plateau phase is added at the end of inspiration while maintaining a constant pressure, i.e. a plateau pressure Pₚₗₐ equal to Pₘₐₓ. In other words, the ventilator 1 then regulates the pressure while maintaining

the maximum pressure Pₘₐₓ, over the course of the time adjusted by the operator, namely the plateau duration (Dp) set by the user.

Then, when the switch is made to regulating pressure, i.e. at the start of the plateau phase, the pressure component linked to the patient's resistance must be preferably quickly compensated for by the regulation system in order to avoid a drop in pressure and discomfort for the patient.

In the case of the use of PID control regulation, the switch from regulating flowrate to regulating pressure requires the resetting of certain parameters, such as the integral factor, to prevent the turbine speed from dropping during the phase of loading the integral factor.

Ideally, the pressure is perfectly maintained at the maximum pressure during this phase and the patient does not feel any transition from one phase to the next.

In general, the respiratory therapy apparatus according to the invention is well suited to the supply of respiratory therapy of the IPPB type to patients who need it, typically to individuals suffering from respiratory problems or conditions that need their bronchial drainage, their respiratory function and their lung recruitment, particularly recruitment of those regions of the lungs that are poorly ventilated or unventilated, to be improved.

What is claimed is:

1. Respiratory therapy apparatus (1) for supplying intermittent positive pressure breathing (IPPB) assistance to an individual, comprising:
a peripheral casing or shell (1.1),
a motorized turbine (2) for supplying a respiratory gas under pressure,
an internal gas circuit (3) for conveying the respiratory gas delivered by the turbine (2),
a flowrate sensor (5) arranged on the internal gas circuit (3),
a nonreturn device (6) arranged in the internal gas circuit (3) downstream of the flowrate sensor (5),
an external patient circuit (10) that can be fluidically connected to the internal gas circuit (3) to convey the respiratory gas coming from the internal gas circuit (3),
a proximal-pressure sensor (7) arranged to allow the measuring of the pressure within the external patient circuit (10), and
operating means (4) connected to the proximal-pressure sensor (7) and to the motorized turbine (2) and configured to command the turbine (2) as a function of at least one pressure measurement taken by the proximal-pressure sensor (7) and of at least one flowrate measurement taken by the flowrate sensor (5),
and wherein the turbine (2), the operating means (4), the internal circuit (3), the flowrate sensor (5) and the nonreturn device (6) are arranged in the peripheral casing or shell (1.1),
characterized in that the operating means (4) are configured to, during each of the individual's inspiratory phases:
a) command the turbine to supply a regulated gas flowrate (Q_{adjusted}), over the course of a non-zero gas insufflation duration (Di) that is sufficient to reach a set maximum-pressure threshold (Pₘₐₓ), and
b) when a measured pressure (Pₘₑₐₛ) measured by the proximal-pressure sensor is greater than or equal to the pre-set maximum-pressure threshold (Pₘₐₓ), command the turbine to supply a plateau pressure (Pₚₗₐₜ) corresponding to the maximum-pressure threshold (Pₘₐₓ) so as to keep at least part of the external patient circuit at said plateau pressure (Pₚₗₐₜ), over the course of a given non-zero plateau duration (Dp).

2. Apparatus according to claim 1, characterized in that it additionally comprises:
an expiratory valve (11) arranged on the external patient circuit (10), and
a pneumatic operating line (8) for operating the expiratory valve (11) fluidically connected (8.1) to the internal gas circuit (3) between the turbine (2) and the flowrate sensor (5), and collaborating with the expiratory valve (11) to control the opening or closing of said expiratory valve (11).

3. Apparatus according to claim 2, characterized in that the proximal-pressure sensor (7) is arranged to enable measurement of the pressure within the external patient circuit (10) via a pressure-measurement line (7.1) that is connected to the patient circuit (10) downstream (7.2) of the expiratory valve (11).

4. Apparatus according to claim 1, characterized in that the operating means (4) are configured to command the turbine (2) such that the gas flowrate (Q_{adjusted}) supplied in step a) is a constant or decelerating flowrate over the course of the gas insufflation duration (Di).

5. Apparatus according to claim 1, characterized in that it comprises duration adjusting means configured to allow the user to adjust the plateau duration (Dp) and/or pressure adjusting means configured to allow the user to adjust the maximum-pressure threshold (Pₘₐₓ).

6. Apparatus according to claim 1, characterized in that the proximal-pressure sensor (7) is configured to enable measurement of the pressure within the external patient circuit (10) between (7.2) the expiratory valve (11) and a breathing interface (20) providing the gas exchanges with the patient.

7. Apparatus according to claim 1, characterized in that, when said operating means (4) determine that the measured proximal pressure (Pₘₑₐₛ) has reached the maximum-pressure threshold (Pₘₐₓ), the operating means (4) are configured to stop operating the turbine (2) to control its flowrate and to start to operate it to control its pressure so as to slow down the turbine (2) and cause a drop in the flowrate of gas supplied.

8. Apparatus according to claim 1, characterized in that the operating means (4) are configured to compare a measured proximal-pressure value (Pₘₑₐₛ) measured by the proximal-pressure sensor (7) with the set maximum-pressure threshold (Pₘₐₓ) and to trigger step b) when the measured pressure value (Pₘₑₐₛ) measured by the proximal-pressure sensor (7) is equal to the pre-set maximum-pressure threshold (Pₘₐₓ).

9. Apparatus according to claim 2, characterized in that, over the course of the plateau duration (Dp), the expiratory valve (11) is closed so as to keep at least part of the external patient circuit (10) at the plateau pressure (Pₚₗₐ).

10. Apparatus according to claim 2, characterized in that, at the end of the plateau duration (Dp), the expiratory valve (11) opens so as to discharge at least some of the gas pressure present in the external patient circuit (10).

11. Apparatus according to claim 1, characterized in that the maximum-pressure threshold (Pₘₐₓ) is comprised between 10 and 50 cmH2O.

12. Apparatus according to claim 1, characterized in that the operating means (4) comprise a timer.

13. Apparatus according to claim 1, characterized in that the operating means (4) comprise at least one microprocessor.

14. Apparatus according to claim 1, characterized in that the motorized turbine (2) comprises an electric motor.

15. Apparatus according to claim 14, characterized in that the operating means (4) are configured to command the accelerations and decelerations or brakings of said electric motor of the turbine (2).
